(19) United States
(12) Patent Application Publication
(10) Pub. No.: US 2016/0296342 A1
(43) Pub. Date: Oct. 13, 2016

(54) FLEXIBLE SPINAL FIXATION DEVICE

(71) Applicant: K2M, Inc., Leesburg, VA (US)

(72) Inventor: Richard W. Woods, Catonsville, MD (US)

(21) Appl. No.: 15/094,112

(22) Filed: Apr. 8, 2016

Related U.S. Application Data
(60) Provisional application No. 62/144,414, filed on Apr. 8, 2015.

(51) Int. Cl. A61F 2/44

(52) U.S. Cl. CPC A61F 2/4455; A61F 2002/4475; A61F 2002/30774

(57) ABSTRACT

A spinal fixation assembly includes an intervertebral body and a fixation member. The intervertebral body is positionable between adjacent vertebral bodies. The intervertebral body includes a front surface, a top surface, and a bottom surface. In particular, the top and bottom surfaces engage the respective adjacent vertebral bodies. The intervertebral body defines a through-hole extending from the front surface toward one of the adjacent vertebral bodies. The fixation member is configured to be received through the through-hole, and includes cut features to facilitate flexing of the fixation member.

### CROSS-REFERENCE TO RELATED APPLICATION

This application claims priority to, and the benefit of, U.S. Provisional Patent Application Ser. No. 62/144,414, filed on Apr. 8, 2015, the entire contents of which are incorporated by reference herein.

### BACKGROUND

1. Technical Field

The present disclosure relates to a spinal fixation device, and more particularly, to a flexible spinal fixation device and a method of use therefor.

2. Background of Related Art

The spine is a flexible structure capable of a large range of motion. There are various disorders, diseases, and types of injury, which restrict the range of motion of the spine or interfere with important elements of the nervous system. The problems include scoliosis, kyphosis, excessive lordosis, spondylolisthesis, slipped or ruptured disc, degenerative disc disease, vertebral body fracture, and tumors. Persons suffering from any of the above conditions typically experience extreme and/or debilitating pain, and often times diminished nerve function.

Spinal fixation apparatuses are widely employed in surgical processes for correcting spinal injuries and diseases. When the disc has degenerated to the point of requiring removal, there are a variety of interbody implants that are utilized to take the place of the disc. These include interbody spacers, metal cages, and cadaver and human bone implants. In order to facilitate stabilizing the spine and keeping the interbody in position, screws or other fixation members are used to connect the intervertebral body with the vertebral bodies.

Therefore, there is a continuing need for a device that can easily and reliably secure the intervertebral body with the vertebral bodies.

### SUMMARY

In accordance with an embodiment of the present disclosure, there is provided a spinal fixation assembly including an intervertebral body and a fixation member. The intervertebral body is positionable between adjacent vertebral bodies. The intervertebral body includes a front surface, a top surface, and a bottom surface. The top and bottom surfaces engage the respective adjacent vertebral bodies. The intervertebral body defines a through-hole extending from the front surface toward one of the adjacent vertebral bodies. The fixation member is configured to be received through the through-hole. The fixation member includes cut features to facilitate flexing of the fixation member. The cut features define a plurality of circumferential sections, wherein each circumferential section of the plurality of circumferential sections includes male and female portions.

The male and female portions may be circumferentially arranged such that the male and female portions are alternately arranged along a length of the fixation member.

In an embodiment, the through-hole of the intervertebral body may define a curvature.

In an embodiment, the fixation member may include a head portion and a shaft extending distally from the head portion. The head portion may include threads to threadably engage the through-hole of the intervertebral body. In addition, the shaft of the fixation member may be bendable transversely with respect to a longitudinal axis of the fixation member. Further, the shaft of the fixation member may include threads configured to threadably engage one of the adjacent vertebral bodies. The shaft of the fixation member may include a retaining member. Furthermore, the retaining member may include a plurality of circumferentially arranged fingers to inhibit proximal displacement of the fixation member. The retaining member of the shaft may be disposed at a distal end of the fixation member.

In an embodiment, the fixation member may be cannulated.

In another embodiment, the spinal fixation assembly may further include a guide wire configured to be received through the fixation member.

In an embodiment, the intervertebral body may include a locking member to threadably secure the fixation member thereto. The fixation member may include threads. In addition, the locking member may include a deformable lip configured to threadably engage the threads of the fixation member.

In yet another embodiment, the head portion of the fixation member may include a first diameter and the shaft of the fixation member may include a second diameter different from the first diameter.

In accordance with another aspect of the present disclosure, there is provided a method of surgery including positioning an intervertebral body between adjacent vertebral bodies; inserting a fixation member through a front surface and into one of the adjacent vertebral bodies; and locking the fixation member to the intervertebral body.

In an embodiment, the method of surgery may further include inserting a flexible drill bit through a through-hole of the intervertebral body and creating a path into one of the adjacent vertebral bodies to receive the fixation member therein.

In another embodiment, inserting the fixation member may include flexing the fixation member such that at least a portion of the fixation member is aligned with a longitudinal axis defined by the adjacent vertebral bodies when the fixation member is inserted through the intervertebral body.

In yet another embodiment, the method of surgery may further include inserting a guidewire through a channel defined in the fixation member.

### BRIEF DESCRIPTION OF THE DRAWINGS

The above and other aspects and features of the present disclosure will become more apparent in light of the following detailed description when taken in conjunction with the accompanying drawings in which:

FIG. 1 is a side view of a fixation member in accordance with an embodiment of the present disclosure;

FIG. 2 is a side cross-sectional view of the fixation member of FIG. 1 taken along line 2-2 of FIG. 1;

FIG. 3 is a perspective view of the fixation member of FIG. 1;

FIG. 4 is a top view of the fixation member of FIG. 1 illustrating the fixation member in a flexed state;

FIG. 5 is a side view of the fixation member of FIG. 4;

FIG. 6a is a bottom view of an intervertebral body and the fixation members of FIG. 1 inserted therein;

FIG. 6b is a side cross-sectional view of the intervertebral body and the fixation members of FIG. 6a taken along line 6b-6b of FIG. 6a;

FIG. 6c is a side view of the intervertebral body and the fixation members of FIG. 6a;

FIG. 7a is a front view of the intervertebral body and the fixation members of

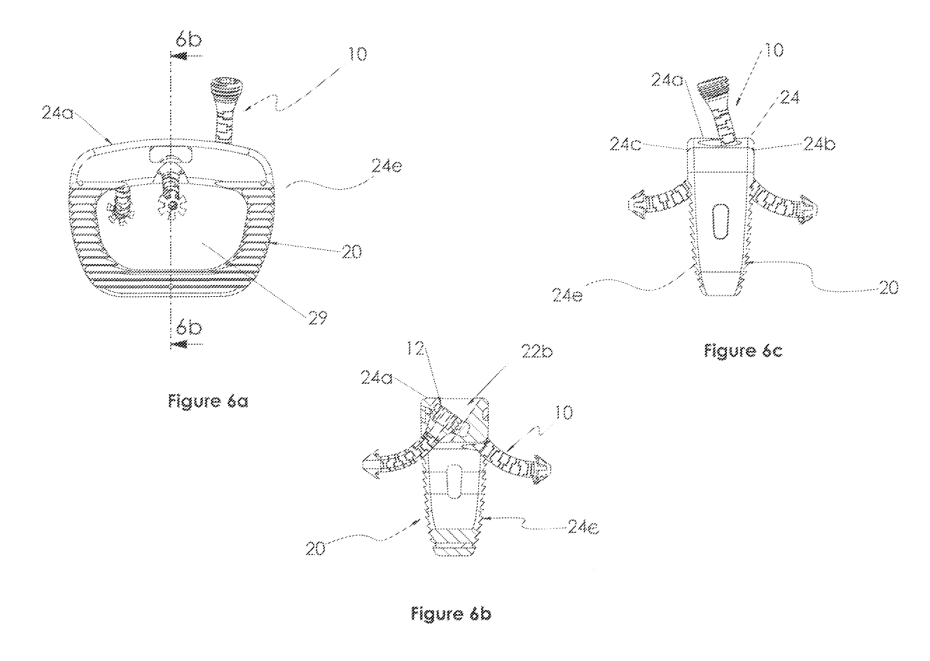

FIG. 6a;

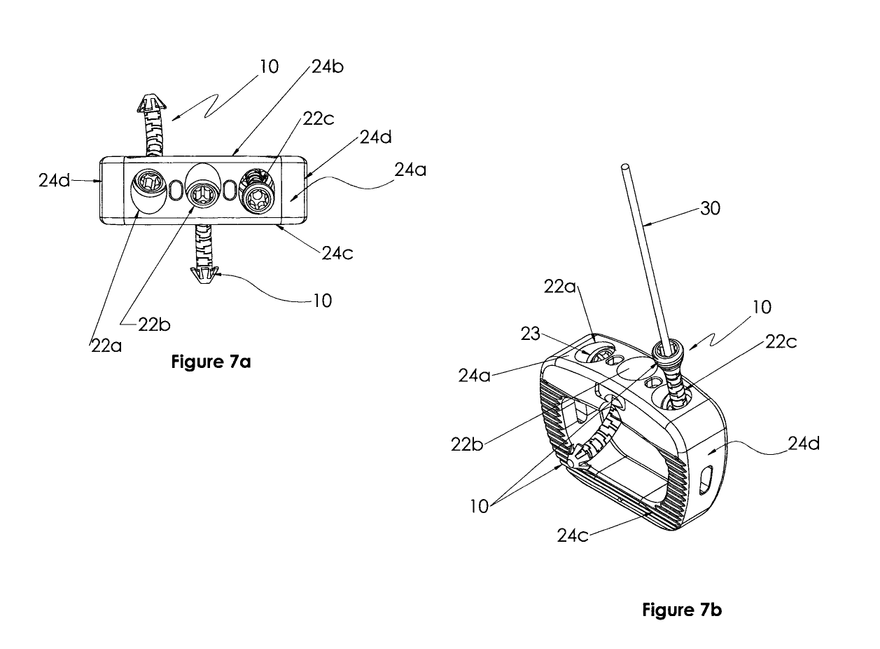

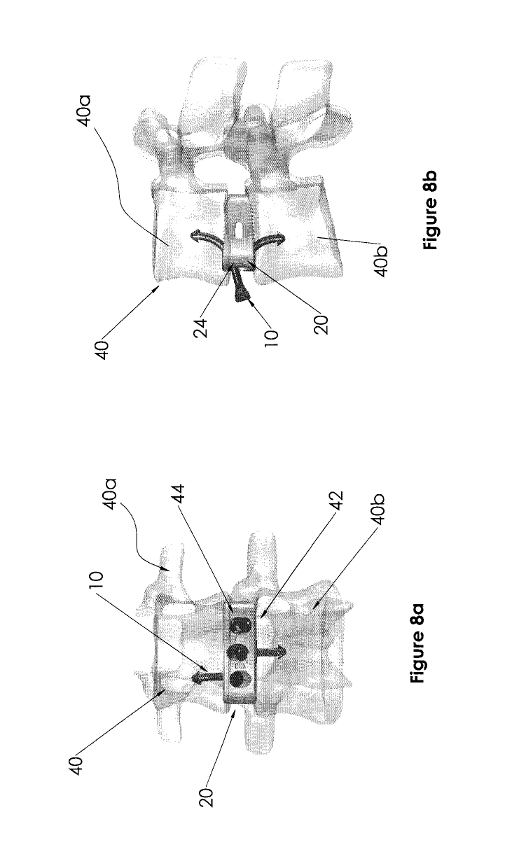

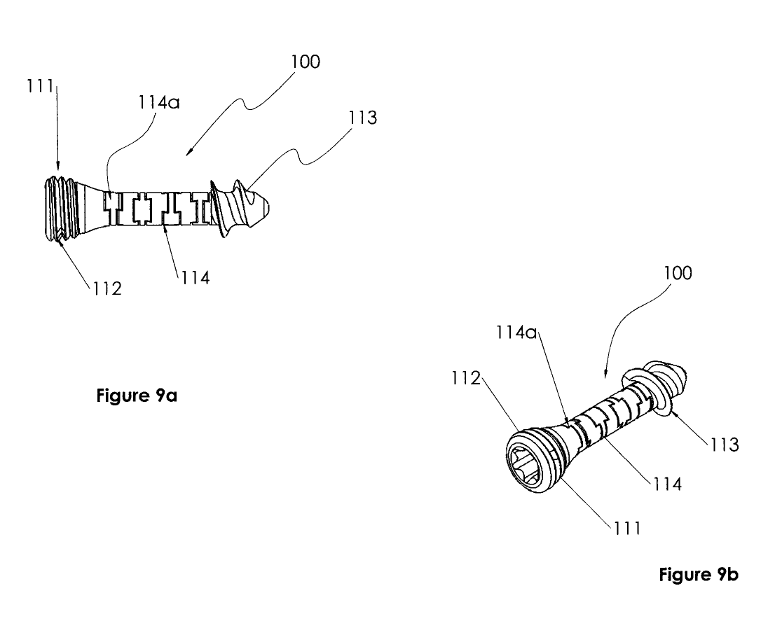

FIG. 7b is a perspective view of the intervertebral body and the fixation members of FIG. 6a illustrating use with a guidewire inserted through one of the fixation members;

FIG. 8a is a front view of the intervertebral body and the fixation members of FIG. 6a illustrating the intervertebral body and the fixation members positioned with vertebral bodies;

FIG. 8b is a side view of the intervertebral body and the fixation members of FIG. 8a;

FIG. 9a is a side view of a fixation member in accordance with another embodiment of the present disclosure; and

FIG. 9b is a perspective view of the fixation member of FIG. 9a.

### DETAILED DESCRIPTION

Particular embodiments of the present disclosure will be described herein with reference to the accompanying drawings. As shown in the drawings and as described throughout the following description, and as is traditional when referring to relative positioning on an object, the terms “proximal” and “trailing” may be employed interchangeably, and should be understood as referring to the portion of a structure that is closer to a clinician during proper use. The terms “distal” and “leading” may also be employed interchangeably, and should be understood as referring to the portion of a structure that is farther from the clinician during proper use.

In addition, the term “cephalad” is used in this application to indicate a direction toward a patient's head, whereas the term “caudad” indicates a direction toward the patient's feet. The term “posterior” indicates a direction toward the patient's back, and the term “anterior” indicates a direction toward the patient's front. Additionally, in the drawings and in the description that follows, terms such as front, rear, upper, lower, top, bottom, and similar directional terms are used simply for convenience of description and are not intended to limit the disclosure. In the following description, well-known functions or constructions are not described in detail to avoid obscuring the present disclosure in unnecessary detail.

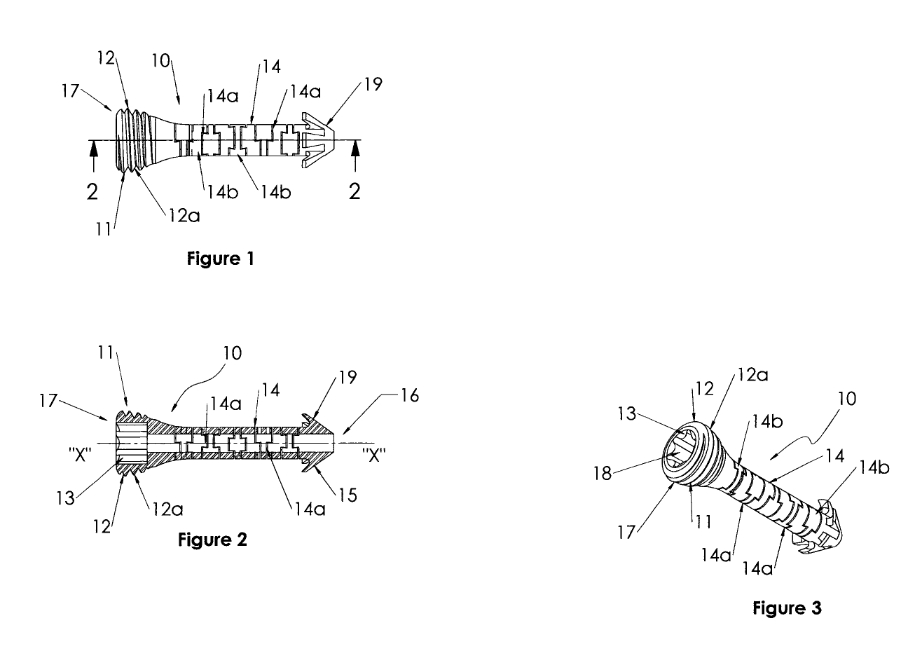

With reference to FIGS. 1-3, an embodiment of the present disclosure is shown generally as a fixation member 10. Fixation member 10 may be used to anchor an intervertebral body 20 (FIG. 6a) positioned between adjacent vertebral bodies 40 (FIG. 8a) or other spinal fixation devices such as, e.g., stabilization plates (not shown). Examples of suitable stabilization plates are disclosed in U.S. Pat. Nos. 8,303,633 and 8,574,272, the entire content of each of which is incorporated herein by reference. However, it is also envisioned that fixation member 10 may be used without intervertebral body 20 or the stabilization plates. Fixation member 10 may be formed of a biocompatible material including, e.g., stainless steel, cobalt chrome, titanium or titanium alloy, and various polymers (PEEK, Radel, PLA, PGA, Ultem, PC, polyethylene, polypropylene, polyacetal or other such engineering resin) or any combinations thereof.

With continued reference to FIGS. 1-3, fixation member 10 includes a head 12 at a proximal end 17 of fixation member 10 and a shaft 14 extending distally from head 12. Shaft 14 has a diameter smaller than a diameter of head 12. An outer surface 12a of head 12 includes threads 11 that threadably engage a lip 23 (FIG. 7b) of a through-hole 22a, 22b, 22c defined in intervertebral body 20. Head 12 of fixation member 10 defines a cavity 13 having, e.g., a hex key feature 18, for non-slip engagement with a driver or other instrument (not shown) to drive fixation member 10 into through-hole 22a, 22b, 22c (FIG. 6b) of intervertebral body 20 and vertebral body 40 (FIG. 8a).

Shaft 14 of fixation member 10 may be selectively bent for the particular surgical application. With brief reference to FIG. 6b, shaft 14 may flex to accommodate, e.g., a curvature, of through-hole 22a, 22b, 22c defined in intervertebral body 20. Shaft 14 is inserted from a front surface 24 (FIG. 7a) of intervertebral body 20. Shaft 14 extends through through-hole 22a, 22b, 22c and into one of adjacent vertebral bodies 40 (FIGS. 8a and 8b). In this manner, a distal end 19 of fixation member 10 is aligned with a longitudinal axis defined by vertebral bodies 40 (FIG. 8a).

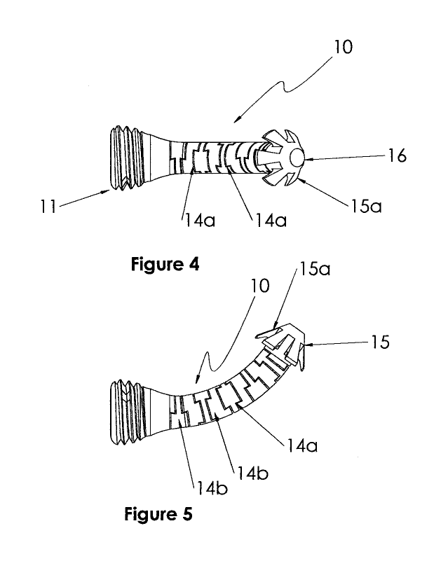

With particular reference now to FIGS. 2-5, fixation member 10 is configured for selective bending or flexing of shaft 14. To this end, shaft 14 includes various cut features 14a to facilitate flexing of shaft 14. Cut features 14a may define a plurality of circumferential sections 14b. Each circumferential section 14b includes male and female portions. The male and female portions are circumferentially arranged such that the male and female portions are alternately arranged along a length of shaft 14. In this manner, shaft 14 may be flexed in various orientations about a longitudinal axis “X-X” (FIG. 2) of fixation member 10. Cut features 14a may include various widths between respective cut features 14a. Cut features 14a may be created by various techniques including, e.g., wire electrical discharge machining, photo etching and the like. In addition, fixation member 10 is cannulated to further facilitate bending of shaft 14. Cannulated fixation member 10 defines a channel 16 dimensioned to receive a guidewire 30 (FIG. 7b) during insertion of fixation member 10.

With continued reference to FIGS. 2-5, distal end 19 of the fixation member 10 includes a retention member 15 configured to engage vertebral bodies 40 and secure fixation member 10 with vertebral bodies 40. Retention member 15 includes a generally tapered configuration. Retention member 15 may be monolithically formed with shaft 14. Retention member 15 includes a plurality of fingers 15a circumferentially arranged and extending radially outward. Each finger 15a is configured to retain fixation member 10 in vertebral bodies 40. In particular, the generally tapered configuration of retention member 15 enables distal displacement of retention member 15 in vertebral bodies 40. However, the plurality of fingers 15a inhibits displacement of fixation member 10 in the proximal direction. In particular, when fixation member 10 is urged in the proximal direction the plurality of fingers 15a in vertebral bodies 40 extend or flex radially outward to inhibit displacement in the proximal direction (FIG. 6a).

With reference now to FIGS. 6a-6c, there is illustrated intervertebral body 20 for use with fixation members 10. Intervertebral body 20 is configured to be positioned between adjacent vertebral bodies 40 (FIGS. 8a and 8b). With reference to FIG. 7a, intervertebral body 20 includes front face 24a, a superior surface 24b, an inferior surface 24c, and side surfaces 24d. Superior and inferior surfaces 24b, 24c are configured to engage respective vertebral bodies 40. Superior and inferior surfaces 24b, 24c may include ridges 24e (FIGS. 6b and 6c). Ridges 24e may provide stability against fore and aft, oblique or side to side movement of intervertebral body 20 within the disc space. Side surfaces 24d extend between superior and inferior surfaces 24b, 24c of vertebral body 40. Intervertebral body 20 defines a cavity 29 adapted for containment of, e.g., bone graft material, to facilitate fusion. In addition, intervertebral body 20 further defines through-holes 22a, 22b, 22c configured to receive fixation member 10. Each through-hole 22a, 22b, 22c extends from front face 24a of intervertebral body 20 toward one of cephalad vertebral body 40a or caudad vertebral body 40b of adjacent vertebral bodies 40. Through-hole 22a, 22b, 22c may define a curvature. With brief reference to FIG. 6b, each through-hole 22a, 22b, 22c may define a curvature toward one of cephalad vertebral body 40a or caudad vertebral body 40b such that at least a portion of fixation member 10 is aligned with a longitudinal axis defined by adjacent vertebral bodies 40. Shaft 14 may flex to accommodate, e.g., the outward curvature, of through-hole 22a, 22b, 22c defined in intervertebral body 20 when shaft 14 is inserted from front surface 24a of intervertebral body 20 and extends toward an inferior surface 44 of the cephalad vertebral body 40a or a superior surface 42 of caudad vertebral body 40b (FIGS. 8a and 8b). In this manner, a distal end 19 of fixation member 10 is, e.g., longitudinally, aligned with vertebral bodies 40 (FIG. 8a).

With continued reference to FIGS. 7a and 7b, first and second through-holes 22a, 22c extend toward superior surface 24b of intervertebral body 20, and a third through-hole 22b extends toward inferior surface 24c of intervertebral body 20. Respective through-holes 22a, 22b, 22c may be curved such that, e.g., distal ends 19, of respective fixation members 10 are longitudinally aligned with a longitudinal axis of vertebral bodies 40 when fixation members 10 are received in respective through-holes 22a, 22b, 22c. It is contemplated that a flexible drill (not shown) may be used to drill bores in adjacent vertebral bodies 40 to create a path to receive fixation member 10 through through-holes 22a, 22b, 22c and into vertebral body 40. In particular, a cannulated flexible drill (not shown) may be used to create a pathway from through-holes 22a, 22b, 22c into cephalad and caudad vertebral bodies 40a, 40b by inserting the cannulated flexible drill over guide wire 30 to guide the drill into vertebral body 40. The drilled path to vertebral bodies 40 facilitates insertion of fixation member 10 with retention member 15 into vertebral body 40. After placing fixation member 10 into through-hole 22a, 22b, 22c of intervertebral body 20, a driver or a tool having, e.g., a hex key feature 18, is utilized to drive threads 11 at proximal end 17 of fixation member 10 into engagement with lip 23 of through-hole 22a, 22b, 22c to fully seat fixation member 10 therewith to lock fixation member 10 to intervertebral body 20.

With particular reference to FIG. 7b, each through-hole 22a, 22b, 22c includes a lip 23 configured to engage threads 11 on outer surface 12a of head 12 of fixation member 10. In particular, threads 11 on head 12 of fixation member 10 are formed from a material having a greater hardness than the material of lip 23, whereby lip 23 is deformed as threads 11 are driven into lip 23, which, in turn, enhances securement of fixation member 10 to intervertebral body 20. Reference may be made to U.S. Pat. No. 6,322,562, filed on Dec. 15, 1999, entitled “Fixation System for Bones” and U.S. Pat. No. 8,137,405, filed Oct. 8, 2008, entitled “Spinal Interbody Spacer,” the entire content of each of which is incorporated herein by reference, for a detailed discussion of the construction and operation of the threadably securing locking mechanism.

Intervertebral body 20 may be formed in whole or in part from any biocompatible material including, e.g., stainless steel, cobalt chrome, titanium or titanium alloy, and various polymers (PEEK, Radel, PLA, PGA, Ultem, PC, polyethylene, polypropylene, polyacetal or other such engineering resin) or any combinations thereof. For example, fixation member 10 may be made of a titanium alloy and intervertebral body 20 may be made of commercially pure titanium. Alternatively, intervertebral body 20 may be made of PEEK, with titanium inserts disposed in slots in the PEEK and defining lip 23, as disclosed in U.S. Pat. No. 8,137,405.

It is further contemplated that intervertebral body 20 may include one or more temporary fixation holes (not shown) configured to receive one or more pins (not shown) to temporarily hold intervertebral body 20 in place relative to adjacent vertebral bodies 40 during drilling and/or placement of fixation members 10.

With reference now to FIGS. 9a and 9b, another embodiment of the present disclosure is shown generally as a fixation member 100. Fixation member 100 is substantially identical to fixation member 10, and thus, identical parts will not be discussed in further detail herein. Fixation member 100 includes a head 112 having threads 111 and a shaft 114 extending distally from head 112. Shaft 114 includes cut features 114a to facilitate bending of the shaft 114. In particular, fixation member 100 includes shaft 114 having a threaded portion 113 configured to threadably engage vertebral bodies 40. Threads 113 may enhance securement of fixation member 100 in vertebral body 40. The pitch and spacing of 113 is selected to permit threads 113 to pass through lip 23 of intervertebral body 20 without deforming lip 23. This may be accomplished by making the maximum diameter of threads 113 smaller than the minimum diameter of lip 23. It is contemplated that fixation member 100 may include a self-starting tip to eliminate the need for prior drilling.

In use, the surgeon may initially perform a discectomy using any known technique. Once the disc space is cleared and the end plates of vertebral bodies 40 are prepared, intervertebral body 20 can be inserted using various means and techniques known in the field including, e.g., anterior lumbar interbody fusion (ALIF), posterior lumbar interbody fusion (PLIF), transforaminal lumbar interbody fusion (TLIF), and lateral insertion methods. Optionally, intervertebral body 20 may be pinned to vertebral body 40 to hold intervertebral body 20 in position during fixation. Optionally, the surgeon may utilize a flexible drill, such as, e.g., a cannulated flexible drill, inserted over a guidewire 30, to create a pathway from through-holes 22a, 22b, 22c of intervertebral body 20 and into the cephalad vertebral body 40a or caudad vertebral body 40b. Guidewire 30 can remain in place and provide a guide for fixation member 10 to follow. An additional inserter instrument (not shown) may be inserted into hex key feature 18 of fixation member 10 to further facilitate insertion of the fixation member 10. Head 12 of fixation member 10 may be positioned to sit flush or be slightly recessed with front surface 24 of the interbody 20 while threads 11 lock with lip 23. Further retention of fixation member 10 in vertebral body 40 is achieved by retention member 15 at distal end 19 of fixation member 10. Retention member 15 or other such retention means may sit flush with the surface of the shaft 14 during insertion and then be deployed once fixation member 10 is in place. Shaft 14 of fixation member 10 may be cylindrical to facilitate rotational insertion of fixation member 10 to engage threads 11 on head 12 of fixation member 10 with lip 23. Alternatively, fixation member 10 may be inserted into vertebral body 40 without pre-drilling if distal end 19 of flexible fixation member is a self-starting sharp tip. Use of fixation member 100 is substantially identical to that of fixation member 10, and thus, will not be described herein.

It will be understood that various modifications may be made to the embodiments disclosed herein. Therefore, the above description should not be construed as limiting, but merely as exemplifications of particular embodiments. Those skilled in the art will envision other modifications within the scope and spirit of the claims appended hereto. For example, it is contemplated that through-hole 22a, 22b, 22c may be straight. It is also contemplated that other screw locking mechanisms such as, e.g., cover plates pre-attached to the implant, cover plates attached to the implant over the screw, tabs engaging screws, cam mechanisms on the implant engaging the screw, or set screws expanding the screw head in the hole, may be utilized. In addition, it is also envisioned that fixation member 10 may be used as part of a pedicle screw, with the fixation member configured to extend through the pedicle and flex within the vertebral body for secure fixation. Under such a configuration, a polyaxial rod receiving head would be attached to the screw head.

It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure.

What is claimed is:

1. A spinal fixation assembly comprising:
an intervertebral body positionable between adjacent vertebral bodies, the intervertebral body including a front surface, a top surface, and a bottom surface, the top and bottom surfaces engaging the respective vertebral bodies, the intervertebral body defining a through-hole extending from the front surface toward one of the adjacent vertebral bodies; and
a fixation member configured to be received through the through-hole, the fixation member including cut features to facilitate flexing of the fixation member, wherein the cut features define a plurality of circumferential sections, each circumferential section of the plurality of circumferential sections including male and female portions.

2. The spinal fixation assembly according to claim 1, wherein the through-hole of the intervertebral body defines a curvature.

3. The spinal fixation assembly according to claim 2, wherein the male and female portions are circumferentially arranged such that the male and female portions are alternately arranged along a length of the fixation member.

4. The spinal fixation assembly according to claim 1, wherein the fixation member includes a head portion and a shaft extending distally from the head portion.

5. The spinal fixation assembly according to claim 4, wherein the head portion includes threads to threadably engage the through-hole of the intervertebral body.

6. The spinal fixation assembly according to claim 4, wherein the shaft of the fixation member is bendable transversely with respect to a longitudinal axis of the fixation member.

7. The spinal fixation assembly according to claim 4, wherein the shaft of the fixation member includes threads configured to threadably engage one of the adjacent vertebral bodies.

8. The spinal fixation assembly according to claim 4, wherein the shaft of the fixation member includes a retaining member.

9. The spinal fixation assembly according to claim 8, wherein the retaining member includes a plurality of circumferentially arranged fingers to inhibit proximal displacement of the fixation member.

10. The spinal fixation assembly according to claim 8, wherein the retaining member of the shaft is disposed at a distal end of the fixation member.

11. The spinal fixation assembly according to claim 8, wherein the fixation member is cannulated.

12. The spinal fixation assembly according to claim 11, further comprising a guide wire configured to be received through the fixation member.

13. The spinal fixation assembly according to claim 1, wherein the intervertebral body includes a locking member to threadably secure the fixation member thereto.

14. The spinal fixation assembly according to claim 13, wherein the fixation member includes threads, and the locking member includes a deformable lip configured to threadably engage the threads of the fixation member.

15. The spinal fixation assembly according to claim 1, wherein the head portion of the fixation member includes a first diameter and the shaft of the fixation member includes a second diameter different from the first diameter.

16. A method of surgery comprising:
positioning an intervertebral body between adjacent vertebral bodies;
inserting a fixation member through a front surface and into one of the adjacent vertebral bodies; and
locking the fixation member to the intervertebral body.

17. The method according to claim 16, further comprising inserting a flexible drill bit through a through-hole of the intervertebral body and creating a path into one of the adjacent vertebral bodies to receive the fixation member therein.

18. The method according to claim 16, wherein inserting the fixation member includes flexing the fixation member such that at least a portion of the fixation member is aligned with a longitudinal axis defined by the adjacent vertebral bodies when the fixation member is inserted through the intervertebral body.

19. The method according to claim 16, further comprising inserting a guidewire through a channel defined in the fixation member.

20. The method according to claim 16, further comprising inserting circumferentially arranged fingers into the one of the adjacent vertebral bodies to inhibit proximal displacement of the fixation member.
